(19) United States
(12) Patent Application Publication
(10) Pub. No.: US 2012/0309763 A1
(43) Pub. Date: Dec. 6, 2012

(54) ANTIFUNGAL COMPOSITION INTENDED TO BE APPLIED TO A PERFORATED NAIL

(75) Inventors: Claire Mallard, Mougins (FR); Nathalie Willcox, Magagnosc (FR)

(73) Assignee: GALDERMA PHARMA S.A., Lausanne 30 Grey (CH)

(21) Appl. No.: 13/516,962

(22) PCT Filed: Dec. 17, 2010
(86) PCT No.: PCT/EP10/70086

§ 371 Date: Aug. 28, 2012

(30) Foreign Application Priority Data
Dec. 18, 2009 (FR) 0959186

(51) Int. Cl. A61K 31/137; A61K 47/18; A61P 31/00; A61K 47/38; A61P 31/10; A61K 31/5375; A61K 47/10

(52) U.S. Cl. 514/239.5; 514/655; 514/784; 514/772; 514/781

(57) ABSTRACT

A pharmaceutical composition intended to be applied to a perforated nail is described. In particular, a composition is descirbed that includes an antifungal agent that is in the form of an acid salt, advantageously a hydrochloride; and a solvent system; and advantageously a cationic or positively charged amphoteric surfactant. The described composition can have a viscosity of less than 500 cPs.

### TECHNICAL FIELD

The present invention relates to the identification of a composition that improves the penetration through a perforated nail of an antifungal agent that is in the form of an acid salt, such as for example terbinafine hydrochloride. Thus, it has been shown that the viscosity of the composition, the presence of a limited amount of volatile solvent, and advantageously the presence of a cationic or amphoteric surfactant such as coco betaine, were factors that promote this penetration through holes in the nail in order to effectively reach the nail bed.

Such pharmaceutical or dermatological compositions are particularly useful for the treatment, in humans and animals, of onychomycoses in particular due to dermatophytes or to Candida.

### PRIOR ART

The nails are frequently the site of onychomycoses especially dermatophytic or candidal onychomycoses.

Although the treatment of these pathologies using antifungal agents is carried out in a favoured manner transungually, the very rigid structure of the nail makes treatment difficult.

During the formation of the nail plate, the basal cells of the matrix grow, the nuclei of the cells break up, the cytoplasms fuse in order to form highly individualized larger keratinized cells that constitute the granular layer.

The nail plate is constituted of dead, keratinized and adherent cells, with no nucleus, but with thick membranes. It essentially contains α-keratin and is made up of three layers: the dorsal and intermediate layers, resulting from the matrix, and the ventral layer, resulting from the nail bed (FIG. 1).

The dorsal part is formed from a few layers of cells rich in hard keratin constituting the granular layer. The intermediate part is constituted of cells rich in keratin that is softer with disappearance of the granular layer, and constitutes three quarters of the total thickness of the nail. The ventral part is formed from one or two layers of cells rich in soft and hyponychial keratin; there is still no granular layer.

The nail is essentially composed of keratin, a scleroprotein rich in sulphur-containing amino acids. From the morphological point of view, keratin fibres are for the most part oriented perpendicular to the growth of the nail, in a plane parallel to the surface of the nail.

The keratin chains are linked together by bonds of various types: hydrogen bond, peptide bond, polar bond and disulphide bond (FIG. 2). These bonds may be attacked by various attacking factors: chemical agents, alkaline agents, oxidizing agents, thioglycolates for the disulphide bridges; by disintegration by strong acids or bases for acid-base unions; or rupture of the hydrogen bridges by water molecules.

The chemical composition of the nail is closer to that of the hair than that of the skin. The fatty lipophilic compounds of the nail represent only 0.1 to 1% of the constituents of the nail. This is essentially cholesterol, which has a plasticizing role.

Water is present between 15 and 18% and may even reach 25%. The water content of the nail is above all dependent on the degree of hygrometry. At saturation, it may reach one third of the dry weight of the nail. It is thus easy to understand that hydrophilic molecules penetrate the nail plate more easily than lipophilic molecules.

There are also trace elements in the nail: zinc, iron, manganese, copper, etc.

Sulphur makes up 5% of the weight of the plate, which is particularly rich in sulphur-containing amino acids, mainly cystine and arginine.

The treatment for onychomycosis comprises various approaches:
surgery, which consists in debriding the lesion down to the healthy nail;
topical antifungal treatments; and
systemic antifungal treatments.

One molecule that is particularly used in this therapeutic field is terbinafine, available in the form of terbinafine hydrochloride (terbinafine HCl) of formula:

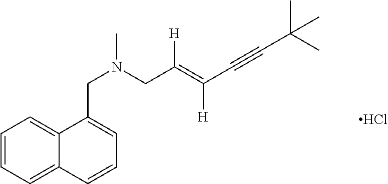

Most oral treatments that have a systemic action are long and are not without significant side effects.

The option of topical treatment proves much less toxic, but in order to be effective, requires the antifungal agent to be able to penetrate through the hard keratin of the nail and reach the nail bed at a sufficient concentration in order to be able to destroy and eradicate the pathogen, Trichophyton rubrum.

However, due to its cationic nature, terbinafine, besides its low solubility in water which gives rise to problems of formulation at high concentration, has a great affinity for keratin. Terbinafine HCl therefore binds very easily to keratin from the upper layers of the nail and due to this fact, only a small proportion actually penetrates down to the nail bed.

In order to overcome this difficulty in passing through the nail, it has been proposed to perforate the nail in order to introduce the active principle close to its site of action.

Thus, document WO 02/11764 proposes to make a multitude of holes in the nail using a laser beam to reach from 80 to 100% of the thickness of the nail.

Document WO 2006/021312 recommends this same system of piercing the nail, but this time to create partial orifices, that is to say that reach from 10 to 80% of the thickness of the nail. A better penetration, for example of the terbinafine, is reported in comparison with an unperforated nail.

However, these documents are not very informative concerning the formulation of the active principles, especially terbinafine, in compositions suitable for this particular mode of application.

Therefore, there is a need to find novel formulations for antifungal agents that allow better penetration through the perforated nail by an effective amount of active principle.

### SUMMARY OF THE INVENTION

Within the context of the present invention, the contribution of the Applicant takes place on various levels:
determining the parameters of a composition so that it is particularly adapted to an application to a perforated nail: suitable consistency, solubilization of the antifungal agent while avoiding crystallization of this agent in the holes of the perforated nail;
showing that making holes promotes the diffusion of the antifungal agents into the various layers of the nail, and especially into the intermediate layer, through the internal lateral surface of the holes. Thus, and to the knowledge of the Applicant, it is the first time that interest has been taken in the diffusion of an active principle such as terbinafine HCl into the various layers of the nail;
demonstrating that the presence of a cationic, advantageously amphoteric, surfactant makes it possible to increase the penetration of an antifungal agent that is in the form of an acid salt, such as terbinafine HCl. Without wishing to be tied to any one theory, it is assumed that the surfactant, due to its cationic or amphoteric nature, binds to the keratin thus enabling the terbinafine HCl to diffuse through the nail matrix and to reach its site of action, the nail bed.

The adapted formulation and the facilitated diffusion of the active principle, directly into the nail bed but also via the intermediate layer, makes it possible to increase the effective concentrations and thus to improve the therapeutic efficacy in the treatment of onychomycosis.

More specifically, the present invention relates to a pharmaceutical composition especially intended to be applied to a perforated nail.

Within the context of the invention and in a favoured manner, the expression “perforated nail” refers to a nail in which openings have been made that must at least perforate the dorsal layer and which may be through-holes, that is to say that open into the nail bed. The purpose is thus to reach at least the intermediate layer, or even the ventral layer too.

Conventionally, it comprises:
an antifungal agent that is in the form of an acid salt, advantageously a hydrochloride; and
a solvent system.

Characteristically in relation to the targeted application, this composition has a viscosity of less than 500 cPs, advantageously less than 400 cPS and preferably between 150 cPs and 300 cPs. The viscosity is measured according to the method described in Example 1, that is to say by using a Brookfield LVDVII+ viscometer equipped with an SC4-18 spindle. The speed and temperature at which the measurements are carried out are respectively 12 rpm and 25° C.

The antifungal agents of interest that are more particularly targeted by the present invention are those from the class of allylamines or morpholines, allylamines being preferred. Indeed, antifungal agents from the class of allylamines, in particular terbinafine or naftitine, and also those from the class of morpholines, in particular amorolfine, are promising compounds in the antifungal fight. Their presumed or demonstrated mode of action appears to take place through inhibition of ergosterol, a specific constituent of the wall of fungal cells, in particular via inhibition of squalene epoxidase.

Within the class of allylamines, mention may be made, in particular, of terbinafine hydrochloride and naftifine hydrochloride, the respective formulae of which are the following:

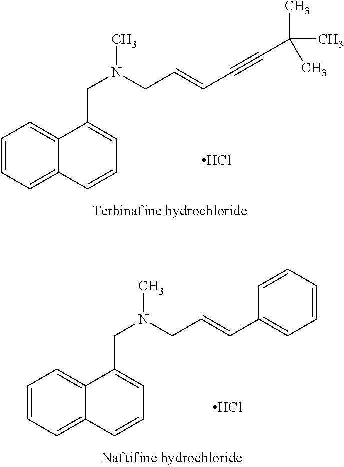

Among the molecules of this class, terbinafine is preferred.

Alternatively, the antifungal agent may belong to the class of morpholines, in particular amorolfine, for which similar problems are faced.

In practice, the antifungal agent such as has been defined above preferably represents more than 5%, or even at least 8%, or even at least 10% (w/w) of the total composition. It is thus possible to envisage up to 15%, or even 20%, of this agent in the composition. Obviously, it is possible to envisage a mixture of antifungal agents, optionally of a different class in order to increase the efficacy.

As already stated, these molecules have the drawback of being almost insoluble in water and of diffusing very little in the nail, probably due to their interaction with keratin.

In practice, it has been determined that one of the important parameters for the viscosity of the composition according to the invention is the solvent system.

Thus, and advantageously, the solvent system of the composition claimed has a content of volatile solvent(s), except for water, of less than or equal to 40% by weight of the total composition.

The expression “volatile solvent” is understood to mean any volatile organic compound defined as being an organic compound having a vapour pressure of 0.01 kPa or more at a temperature of 293.15 K or having a corresponding volatility under the particular usage conditions.

An organic compound is defined as a compound containing at least the element carbon and one or more of the following elements: hydrogen, halogen, oxygen, sulphur, phosphorus, silicon or nitrogen, except for oxides of carbon and inorganic carbonates and bicarbonates.

More advantageously still, the solvent system is a ternary aqueous system constituted of:
water;
at least one C2-C8 alkanol with a straight or branched chain, advantageously ethanol; and
at least one glycol (having free hydroxyl functional groups), advantageously propylene glycol.

In this system, the volatile solvent corresponds to the alkanol, advantageously ethanol, which should therefore represent less than 40% by weight of the composition.

More advantageously still, the amount of total water represents more than 30% by weight of the composition, advantageously more than 33%, or more than 35% or even more than 40%. This large amount of water in the formula gives the product a considerable hydrophilic nature. Indeed, since the nail is a hygroscopic hydrophilic matrix, it swells in the presence of water which facilitates the diffusion of the active principles.

The expression “total water” is understood to mean the amount of water introduced as is into the composition, added to the amount of water originating from the various solvents and/or excipients of the composition when they contain some thereof.

This high water content is extremely advantageous due to the targeted transungual application. Moreover, it has been shown within the context of the invention that, despite the low solubility in water of the antifungal molecules in question, the solvent system proposed allows a solubilization at high concentration of the molecules of interest, including in the presence of the cationic or amphoteric surfactant according to the invention.

Besides the high water content, the ternary solvent system advantageously contains a short-chain alcohol, and more precisely at least one C2-C8 alkanol having a straight or branched chain, preferably ethanol, isopropanol and n-butanol. Ethanol is particularly preferred. A mixture of various alcohols may also be envisaged.

Finally, this ternary solvent system comprises at least one glycol. The term “glycol” is understood here to mean a compound that has at least two hydroxyl functional groups. Glycols for which the two hydroxyl functional groups are free, that is to say that they are not involved in an ether or ester bond, are more precisely targeted by the invention. Mention may be made, for example, of propylene glycol, butylene glycol, hexylene glycol, ethylene glycol and polyethylene glycols. Propylene glycol is preferred. A mixture of various glycols may also be envisaged.

Advantageously, the ternary solvent system represents at least 60%, or 70%, 80% or even 90% (w/w) of the total composition.

In addition, and advantageously, the proportion of alcohol is greater than or equal to that of glycol. More advantageously still, the proportion of total water is greater than that of glycol.

According to one favoured embodiment, the composition also contains a surfactant of cationic, or even amphoteric, nature capable of entering into competition with the antifungal agent in the form of an acid salt for binding to keratin, which is present in the structure of the various layers of the nail in the form of fibres and which is negatively charged.

Indeed, the composition claimed is intended to be applied to holes made in the nail. Besides a more direct access to the nail bed, these holes allow, via their side walls, a diffusion of the active principle—in this case an antifungal agent that is in the form of an acid salt—into the various layers of the nail, and in particular into the intermediate layer. However, as demonstrated in the present application, the presence of a cationic or amphoteric surfactant increases in situ the bioavailability of the active principle in the nail, especially of terbinafine HCl, through the keratinized ungual nail plate, skin appendages and skin.

By definition, a cationic surfactant is a surfactant in which the hydrophilic part is positively charged. It releases a positive charge (cation) in aqueous solution. It has bacteriostatic and emulsifying properties and exhibits an affinity with the negatively charged keratin with which it will combine.

An amphoteric surfactant is a surfactant in which the hydrophilic part comprises a positive charge and a negative charge, the overall charge being zero. Depending on the pH of the medium which it is in, it releases a positive ion and a negative ion. At alkaline pH, it behaves as an anionic surfactant, and at acid pH, it behaves as a cationic surfactant.

Within the context of the invention, in so far as the antifungal agent is in the form of an acid salt, the composition is therefore at acid pH below the pKa of the antifungal agent, preferably at a pH between 3 and 6, preferentially between 3 and 5, and the positively charged amphoteric surfactant then acts as a cationic surfactant.

As cationic surfactants that can be used according to the invention, mention will be made, non-limitingly, of:
quaternary ammoniums,
in which the counterion may be
   chloride, bromide, phosphate, hydroxide, methosulphate, sulphate or a carboxylic acid anion;
in which the substituents of the nitrogen may be
   saturated or unsaturated, optionally hydroxylated, alkyl chains having 1 to 20 carbons, the hydroxyl functional group possibly being esterified, it being possible for these chains to optionally be substituted, to originate from defined compounds, or else to be mixtures resulting from natural products;
   optionally substituted aromatic groups, rings, in particular aromatic rings, for example pyridine, which are optionally substituted;
   mixtures of these various categories;
   themselves substituted by a quaternized or non-quaternized amine functional group;
amines, which may be protonated depending on the pH and amine salts in which the nitrogen bears the substituents mentioned previously and/or hydrogen, these products being used under conditions where they are cationic;
derivatives of betaine or of amino acids, under pH conditions that make them cationic, optionally substituted by the groups mentioned previously.

Advantageously, the amphoteric surfactants used have a structure of a betaine derivative corresponding to the general formula (I) below:

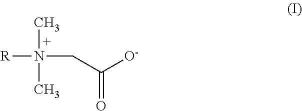

in which R represents an alkyl radical or an R′CO—NH(CH₂)₃— radical, R′ representing an alkyl radical.

The expression “alkyl radical” is understood to mean a saturated, linear or branched hydrocarbon-based chain. Among the alkyl radicals, those comprising from 1 to 20 carbon atoms will be preferred.

Among the betaine derivatives, preference will more particularly be given to the amphoteric surfactant known under the trade name Dehyton® AB30 or else Lauryl

Dimethylaminoacetic Acid Betaine corresponding to the general formula (I) in which R represents the lauryl radical. This molecule is commonly known as coco betaine.

This molecule is, for example, sold by Cognis as a 30% aqueous solution under the name Dehyton® AB30.

As additional examples, mention may be made of other betaines such as cetyl betaine or else cocamidopropyl betaine.

Within the context of the invention and advantageously, the cationic or amphoteric surfactant represents at least 0.1% by weight of the composition. It may represent up to 10%, or even 15% by weight of the composition without disturbing the solubilization of the antifungal agent at high concentration. Typically it represents from 0.1 to 20% by weight of the composition, advantageously from 0.1 to 15%, more advantageously still from 0.1 to 10%.

According to one particular embodiment, the cationic and/or amphoteric surfactant is the sole surfactant in the composition according to the invention. This excludes the simultaneous presence of non-ionic surfactants and also of anionic surfactants.

The fact of using an amphoteric surfactant gives the composition, as another advantage, a washable aspect. Specifically, by simple rinsing with water, it is possible to remove the composition. This is particularly advantageous in the case of application to a perforated nail.

Advantageously, the composition according to the invention also comprises a texturing agent from the class of celluloses, for example alkyl cellulose derivatives, in particular methyl celluloses, ethyl celluloses, propyl celluloses and hydroxyalkyl celluloses, such as those sold under the name KLUCEL, advantageously hydroxyethyl cellulose (Natrosol HHX250) or hydroxypropyl cellulose. This texturing agent makes it possible to adjust the viscosity of the composition and thus to achieve values suitable for application to a perforated nail, as mentioned above. Moreover, the control of the viscosity makes it possible to avoid the too rapid evaporation of the solvents, which makes it possible to avoid, inter alia, obstruction of the holes made in the nail via recrystallization of the active principle.

Within the context of the invention and advantageously, the texturing agent represents at least 0.1% by weight of the composition. It may represent up to 1%, or even 2% by weight of the composition. Typically it represents from 0.1 to 1% by weight of the composition, advantageously from 0.1 to 0.5%, more advantageously still from 0.3 to 0.5%.

Furthermore, the composition according to the invention may also contain at least one additive chosen from the group formed by:
preservatives, such as phenyl ethyl alcohol, benzyl alcohol, phenoxyethanol, parabens and derivatives thereof;
antioxidants, such as butylhydroxyanisole (BHA), butylhydroxytoluene (BHT), palmityl ascorbate, α-tocopherol and/or its esters;
dyes, fillers or pigments, such as the titanium micas commonly used in the cosmetics field for producing nail varnishes;
chelating agents such as disodium EDETATE (EDTA);
emollients such as cyclomethicone; and
other active principles such as an antiseptic, especially acetic acid.

The amounts of each of these additives are easily determined by a person skilled in the art.

Advantageously, the composition according to the invention is an aqueous composition of solution type. The term “solution” is understood to mean a clear and homogeneous liquid preparation containing one or more substances dissolved in a solvent or mixture of solvents that are miscible with one another. The expression “liquid preparation” is understood to mean a product which flows at room temperature and that has a Newtonian character or exhibits pseudoplastic flow. In one favoured embodiment, the composition is in the form of a gelled solution that can be rinsed with water.

Typically, and by way of illustration, a composition according to the invention comprises:
1% to 20% by weight of terbinafine HCl in solubilized form;
0% to 10% by weight of a texturing agent;
0.1% to 20% by weight of an amphoteric surfactant;
20% to 80% by weight of solvent phase predominantly containing water;
0% to 1% of a chelating agent;
0% to 2% of an antioxidant; and
0% to 20% of additives.

Preferably, the composition is constituted of:
1% to 15% by weight of terbinafine HCl in solubilized form;
0% to 5% by weight of a texturing agent;
0.1% to 15% by weight of an amphoteric surfactant;
20% to 60% by weight of solvent phase predominantly containing water;
0% to 0.5% of a chelating agent;
0% to 1% of an antioxidant; and
0% to 10% of additives.

More preferably still, the composition is constituted of:
1% to 10% by weight of terbinafine HCl in solubilized form;
0% to 2% by weight of a texturing agent;
0.1% to 10% by weight of an amphoteric surfactant;
20% to 55% by weight of solvent phase predominantly containing water;
0% to 0.05% of a chelating agent;
0% to 0.5% of an antioxidant; and
0% to 5% of additives.

Preferably, the composition is constituted of:
1% to 10% by weight of terbinafine HCl in solubilized form;
0.1% to 2% by weight of a texturing agent;
0.1% to 10% by weight of an amphoteric surfactant;
20% to 55% by weight of solvent phase predominantly containing water;
0% to 0.05% of a chelating agent;
0% to 0.5% of an antioxidant; and
0% to 5% of additives.

Thus, the present invention relates to a pharmaceutical or dermatological composition intended for the treatment of onychomycoses.

As already stated, a composition according to the invention is particularly suitable for the application thereof to perforated nails.

Since it is difficult to take measurements of the thickness of the nail and of its various layers, it is recommended to make holes having a depth between 10% and 100% of the total thickness of the nail. In practice, the holes have a depth typically between 0.2 and 5 mm (in the particular case of nails affected by onychomycosis).

Advantageously, these holes have a diameter between 400 μm and 1 mm and more particularly between 400 μm and 600 μm. They advantageously have a cylindrical or conical shape.

Various technologies are available for creating such holes. The use of laser beams is for example described in documents WO 02/11764 and WO 2006/021312. Alternatively, a device of drill type, for instance that described in document WO 2004/086938, may be used.

The composition according to the invention can be applied into the holes using pipettes or syringes. The volumes deposited into each hole are typically between 0.05 and 2 μl. A protective layer may optionally be deposited on the surface of the treated nail.

### Exemplary Embodiments

The invention and its attendant advantages will emerge more clearly from the following exemplary embodiments, in support of the appended figures. These are not however in any case limiting.

### KEY TO THE FIGURES

FIG. 1 represents a cross-sectional diagram of the structure of a nail.

FIG. 2 is a schematic representation of the various bonds that exist in the keratin chains in the nail.

FIG. 3 represents the amount of terbinafine accumulated in the receiving liquid (ng/cm²) through the perforated nails in the case of the amphoteric solution and various commercial forms containing terbinafine.

FIG. 4 represents the amount (ng/cm²) of terbinafine accumulated after 5 days of application to the nail in the case of the amphoteric solution and various commercial forms containing terbinafine.

FIG. 5 represents the amount of terbinafine accumulated in the receiving liquid (μg/cm²) through the perforated nails in the case of the amphoteric solution and various commercial forms containing terbinafine, with the amphoteric solution taken as reference.

FIG. 6 represents the amount of terbinafine accumulated in the receiving liquid (μg/cm²) through the perforated nails in the case of a solution with or without amphoteric agent.

FIG. 7 represents a cross-sectional diagram of a hole in the nail revealing the coloured zones to be considered and that to be excluded for the evaluation of the diffusion.

FIG. 8 represents the amount of terbinafine accumulated in the receiving liquid, relative to the thickness of the nails, (ng/cm²/mm) after 5 days of application to the intermediate and dorsal layers, in the case of a solution with or without amphoteric agent.

FIG. 9 represents the amount of terbinafine in the nail (ng/mg) after 5 days of application to the intermediate and dorsal layers, in the case of a solution with or without amphoteric agent.

FIG. 10 represents the amount of terbinafine in the nail (ng/mg) after 5 days of application to the intermediate and dorsal layers, in the case of a Lamisil Spray® type solution with or without amphoteric agent.

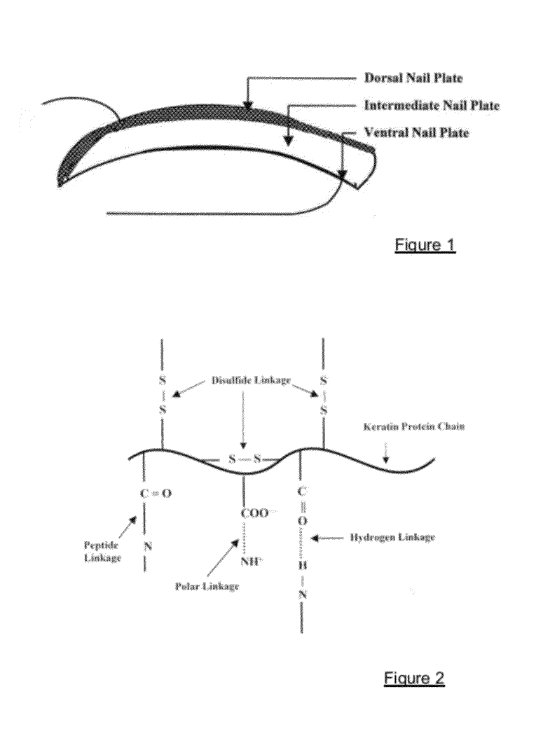

### EXAMPLE 1

### Process for Manufacturing a Composition Based on Terbinafine HCl Containing a Cationic or Amphoteric Surfactant and Study of the Stability of Said Composition

1/Manufacturing Process:

This manufacturing process is carried out simply in a manufacturing beaker by swelling the texturing agent in water. Next the active phase (see preparation below) containing the terbinafine hydrochloride in solution is added. Then, under moderate stirring, the cationic or amphoteric surfactant is added.

a—Preparation of the Aqueous Phase:

Water and the texturing agent are introduced into a beaker and left under stirring in order to obtain a clear, smooth and homogeneous mixture.

b—Preparation of the Active Phase:

In an additional beaker, the active principle is solubilized in the organic, glycol and alcoholic solvents.

c. Final Mixture:

The active phase (b) is incorporated into the aqueous phase (a) and homogenized, then the cationic or amphoteric surfactant is added and the homogenization is continued. A composition based on terbinafine HCl containing a cationic or amphoteric surfactant is thus obtained.

### Viscosity Control Method:

Brookfield LVDVII+ viscometer
SC4-18+small volume spindle
Rate: 12 rpm
Time: 1 minute
Temperature: 25° C.

2/Measurement of the Stability of the Composition:

a—Physical Stability:

The physical stability of the formulations is measured by a macroscopic observation of the formulation at room temperature (RT), at 4° C. and at 40° C., after 1 month, 2 months and 3 months in order to guarantee the physical integrity of the products and to verify the absence of recrystallization of the solubilized terbinafine HCl.

Microscopic analysis is carried out at 4° C. and at room temperature (RT) in order to verify the absence of recrystallization of the solubilized terbinafine HCl.

b—Chemical Stability:

The chemical stability is measured by assaying the active principle using HPLC and the results are expressed as % of the initial content.

### EXAMPLE 2

### Amphoteric Solution Containing 10% of Terbinafine HCl

| | | Content (% taking into |
|---|---|---|
| | Content | account the water contained |
| Ingredients/INCI name | (% w/w) | in each ingredient) |
| Purified water | 27.87 | / |
| Total water (purified water + | / | 27.87 + (6.0 × 0.7) + |
| water contained in the | | (35.53 × 0.05) = 33.85 |
| ingredients) | | |
| Titriplex III/disodium EDTA | 0.01 | 0.01 |
| Natrosol 250 HHX/Hydroxy- | 0.30 | 0.30 |
| ethylcellulose | | |
| Dehyton AB30 (30% aqueous | 6.0 | / |
| solution)/Coco-betaine | | |
| Coco betaine active matter | / | 6.0 × 0.3 = 1.8 |
| (without water) | | |
| Propylene Glycol | 20.25 | 20.25 |
| Ethanol 95-96% | 35.53 | / |
| Absolute ethanol | / | 35.53 − (35.53 × |
| | | 0.05) = 33.75 |
| Nipanox BHT/Butyl hydroxy- | 0.04 | 0.04 |
| toluene | | |
| Terbinafine HCl | 10.0 | 10.0 |

The initial pH is 4.69.

### Physical Stability:

| | | | Initial time | | |
|---|---|---|---|---|---|
| | | | 1 month | 2 months | 3 months |
| Macroscopic | Colourless | 4° C. | Idem | Idem | Idem |
| appearance | clear fluid | | initial | initial | initial |
| | solution | | time | time | time |
| | | RT | Idem | Idem | Idem |
| | | | initial | initial | initial |
| | | | time | time | time |
| | | 40° C. | Idem | Idem | Idem |
| | | | initial | initial | initial |
| | | | time | time | time |
| Microscopic | Absence of | 4° C. | Idem | Idem | Idem |
| appearance | crystals | | initial | initial | initial |
| | | | time | time | time |
| | | RT | Idem | Idem | Idem |
| | | | initial | initial | initial |
| | | | time | time | time |

### Chemical Stability:

| | T0 | T 1 month | T 2 months | T 3 months |
|---|---|---|---|---|
| RT | 100.8% | 102.2% | 101.2% | 99.3% |
| 40° C. | — | 101.8% | 101% | 100.1% |

The composition from Example 2 is therefore physically and chemically stable over 3 months at 4° C., at room temperature and at 40° C.

### EXAMPLE 3

### Amphoteric Solution Containing 10% of Terbinafine HCl

| | | Content (% taking into |
|---|---|---|
| | Content | account the water contained |
| Ingredients/INCI name | (% w/w) | in each ingredient) |
| Purified water | 29.45 | / |
| Total water (purified water + | / | 29.45 + (6.0 × |
| water contained in the | | 0.7) = 33.65 |
| ingredients) | | |
| Titriplex III/disodium EDTA | 0.01 | 0.01 |
| Klucel HF/Hydroxypropyl- | 0.5 | 0.5 |
| cellulose | | |
| Dehyton AB30 (30% aqueous | 6.0 | / |
| solution)/Coco-betaine | | |
| Coco betaine active matter | / | 6.0 × 0.3 = 1.8 |
| (without water) | | |
| Propylene Glycol | 20.25 | 20.25 |
| Absolute ethanol | 33.75 | 33.75 |
| Nipanox BHT/Butyl hydroxy- | 0.04 | 0.04 |
| toluene | | |
| Terbinafine HCl | 10.0 | 10.0 |

The initial pH is 4.50.

### Physical Stability:

| | | | Initial time | | |
|---|---|---|---|---|---|
| | | | 1 month | 2 months | 3 months |
| Macroscopic | Colourless | 4° C. | Idem | Idem | Idem |
| appearance | clear fluid | | initial | initial | initial |
| | solution | | time | time | time |
| | | RT | Idem | Idem | Idem |
| | | | initial | initial | initial |
| | | | time | time | time |
| | | 40° C. | Idem | Idem | Idem |
| | | | initial | initial | initial |
| | | | time | time | time |
| Microscopic | Absence of | 4° C. | Idem | Idem | Idem |
| appearance | crystals | | initial | initial | initial |
| | | | time | time | time |
| | | RT | Idem | Idem | Idem |
| | | | initial | initial | initial |
| | | | time | time | time |

### Chemical Stability:

| | T0 | T 1 month | T 2 months | T 3 months |
|---|---|---|---|---|
| RT | 104% | 103.8% | 102.1% | 103.5% |
| 40° C. | — | 103.6% | 103% | 103.4% |

The composition from Example 3 is therefore physically and chemically stable over 3 months at 4° C., at room temperature and at 40° C.

### EXAMPLE 4A

### Solution Containing 10% of Terbinafine

| | | | Content (% taking |
|---|---|---|---|
| | | | into account the |
| | | Content | water contained in |
| Ingredients | INCI name | (% w/w) | each ingredient) |
| Purified water | Aqua | 33.495 | / |
| Total water | Aqua | / | 33.495 + (0.375 × |
| (purified water + | | | 0.7) + (35.53 × |
| water contained in | | | 0.05) = 35.54 |
| the ingredients) | | | |
| Titriplex III | Disodium | 0.01 | 0.01 |
| | EDTA | | |
| Natrosol 250 HHX | Hydroxyethyl- | 0.30 | 0.30 |
| | cellulose | | |
| Dehyton AB30 (30% | Coco betaine | 0.375 | / |
| aqueous solution) | | | |
| Coco betaine active | Coco betaine | / | 0.375 × 0.3 = 0.11 |
| matter (without | | | |
| water) | | | |
| Propylene | Propylene | 20.25 | 20.25 |
| glycol | glycol | | |
| Ethanol 95-96% | Ethanol | 35.53 | / |
| Absolute ethanol | Ethanol | / | 35.53 − (35.53 × |
| | | | 0.05) = 33.75 |
| Nipanox BHT | Butyl hydroxy- | 0.04 | 0.04 |
| | toluene | | |
| Terbinafine | Terbinafine | 10.0 | 10.0 |
| HCl | HCl | | |

### EXAMPLE 4B

### Solution Containing 10% of Terbinafine

| | | | Content (% taking |
|---|---|---|---|
| | | | into account the |
| | | Content | water contained in |
| Ingredients | INCI name | (% w/w) | each ingredient) |
| Purified water | Aqua | 33.537 | / |
| Total water | Aqua | / | 33.537 + (0.333 × |
| (purified water + | | | 0.7) + (35.53 × |
| water contained in | | | 0.05) = 35.42 |
| the ingredients) | | | |
| Titriplex III | Disodium | 0.01 | 0.01 |
| | EDTA | | |
| Natrosol 250 HHX | Hydroxyethyl- | 0.30 | 0.30 |
| | cellulose | | |
| Dehyton AB30 (30% | Coco-betaine | 0.333 | / |
| aqueous solution) | | | |
| Coco betaine active | Coco-betaine | / | 0.333 × 0.3 = 0.1 |
| matter (without | | | |
| water) | | | |
| Propylene | Propylene | 20.25 | 20.25 |
| glycol | glycol | | |
| Ethanol 95-96% | Ethanol | 35.53 | / |
| Absolute ethanol | Ethanol | / | 35.53 − (35.53 × |
| | | | 0.05) = 33.75 |
| Nipanox BHT | Butyl hydroxy- | 0.04 | 0.04 |
| | toluene | | |
| Terbinafine | Terbinafine | 10.0 | 10.0 |
| HCl | HCl | | |

### EXAMPLE 5

### Solution Containing 10% of Terbinafine

| | | | Content (% taking |
|---|---|---|---|
| | | | into account the |
| | | Content | water contained in |
| Ingredients | INCI name | (% w/w) | each ingredient) |
| Purified water | Aqua | 32.37 | / |
| Total water | Aqua | / | 32.37 + (1.50 × |
| (purified water + | | | 0.7) + (35.53 × |
| water contained in | | | 0.05) = 35.20 |
| the ingredients) | | | |
| Titriplex III | Disodium | 0.01 | 0.01 |
| | EDTA | | |
| Natrosol 250 HHX | Hydroxyethyl- | 0.30 | 0.30 |
| | cellulose | | |
| Dehyton AB30 (30% | Coco betaine | 1.50 | / |
| aqueous solution) | | | |
| Coco betaine active | Coco betaine | / | 1.50 × 0.3 = 0.45 |
| matter (without | | | |
| water) | | | |
| Propylene | Propylene | 20.25 | 20.25 |
| glycol | glycol | | |
| Ethanol 95-96% | Ethanol | 35.53 | / |
| Absolute ethanol | Ethanol | / | 35.53 − (35.53 × |
| | | | 0.05) = 33.75 |
| Nipanox BHT | Butyl hydroxy- | 0.04 | 0.04 |
| | toluene | | |
| Terbinafine | Terbinafine | 10.0 | 10.0 |
| HCl | HCl | | |

### EXAMPLE 6

### Study of the Diffusion of Terbinafine Through Perforated Nails

The objective of this study is to evaluate the penetration of various commercial formulations containing terbinafine HCl through the perforated nails of human cadavers and to compare this penetration with that obtained with an amphoteric solution as prepared according to the process described in Example 1.

For this study, the nails were completely perforated by 3 holes having a diameter of 0.6 mm. The total treatment time was 5 days with a new application of each formulation every day. The application is 10 μl·cm⁻² or 10 mg·cm⁻² depending on the formulation.

At the end of the treatment, the withdrawals were analysed in order to determine the in vitro penetration of the terbinafine through and into the nails.

The formulations tested are the following:
10% amphoteric solution corresponding to Example 2
1% Lamisil Spray
1% Lamisilate Monodose
1% Lamisil Gel
1% Lamisil Cream

The commercial formulations have the following qualitative and quantitative compositions:

| 1% Lamisilate Monodose | | 1% Lamisil AT Spray | |
|---|---|---|---|
| | Content | | Content |
| Ingredients | (% w/w) | Ingredients | (% w/w) |
| Terbinafine HCl | 1 | Terbinafine HCl | 1 |
| Ethanol 96% | 86.37 | Purified water | N/A |
| Acrylate/octylacryl- | N/A | Ethanol 96% | 10 |
| amide copolymer | | | |
| Hydroxypropyl | N/A | Propylene glycol | N/A |
| cellulose | | | |
| Medium chain | N/A | Cetomacrogol | N/A |
| triglycerides | | 1000 | |

N/A: not available

| 1% Lamisil Gel | | 1% Lamisil Cream | |
|---|---|---|---|
| | Content | | Content |
| Ingredients | (% w/w) | Ingredients | (% w/w) |
| Terbinafine Base | 1 | Terbinafine HCl | 1 |
| Purified water | N/A | Purified water | N/A |
| Ethanol 96% | 10 | Sodium hydroxide | N/A |
| Isopropyl myristate | N/A | Benzyl alcohol | N/A |
| Polysorbate 20 | N/A | Sorbitan stearate | N/A |
| Carbomer 974P | N/A | Cetyl stearyl alcohol | N/A |
| Sorbitan laurate | N/A | Polysorbate 60 | N/A |
| Benzyl alcohol | N/A | Isopropyl myristate | N/A |
| Sodium hydroxide | N/A | | |
| Butyl hydroxytoluene | N/A | | |

N/A: not available

The viscosities of the various formulations tested were measured using a Brookfield LVDVII+ viscometer at 25° C.

| Formulations | Spindle | Rate | Viscosity (cPs) |
|---|---|---|---|
| 1% Lamisil Cream | SC4-25 | 12 rpm | 23 715 |
| 1% Lamisilate Monodose | SC4-25 | 12 rpm | 17 916 |
| 1% Lamisil Gel | SC4-25 | 1.5 rpm | 289.10³ |
| 1% Lamisil Spray | SC4-18 | 12 rpm | 3.75 |
| 10% amphoteric solution | SC4-18 | 12 rpm | 205 |

### A/ Materials and Method:

Diffusion cells of Franz type were used to study the penetration of terbinafine through the nail perforated with 3 holes. The piercing was carried out manually at the centre of the nail with holes spaced around 0.2 mm apart.

During the five days of experimentation, the diffusion cells were placed in a thermostatically-controlled bath at 34.2° C. in order to maintain a temperature of 32±1° C. at the surface of the nails.

Every 24 hours, all of the receiving liquid of the diffusion cells containing phosphate buffer, pH 7.4±0.1 and 0.1% of Volpo (Oleth-20) was withdrawn and replaced with an equivalent volume. The surface of the nails was gently cleaned before each reapplication. After the last withdrawal of receiving liquid, the nails were treated in order to extract the terbinafine present in the keratin matrix.

All the withdrawals, receiving liquids and nail extracts were assayed by HPLC (quantification limit 20 ng/ml).

### B/ Results:

In the course of the study, a systematic observation of the holes, before the withdrawal of the receiving liquid and after the cleaning of the surface of the nail, was carried out in order to note the presence or absence of blockages obstructing the holes.

| | Amphoteric | Lamisil | Lamisil | Lamisil | Lamisil |
|---|---|---|---|---|---|
| | solution | Spray | Monodose | Gel | Cream |
| | (10%) | (1%) | (1%) | (1%) | (1%) |
| Before | 0/180 * | 0/90 ** | 66/90 | 54/90 | 76/90 |
| withdrawal | | | | | |
| Before new | 0/180 | 0/90 | 6/90 | 2/90 | 19/90 |
| application | | | | | |

* 180 corresponds to 2 series of 3 holes × 6 nails × 5 applications
** 90 corresponds to 3 holes × 6 nails × 5 applications

The amphoteric solution and Lamisil Spray formulations, which are the most fluid, never obstructed the holes. With the Lamisilate Monodose and Lamisil Gel formulations, between 60 and 75% of the holes were blocked. However, during the cleaning and rinsing step, most of these holes were unblocked since no more than 7% of holes remained blocked. The Lamisil Cream formula was the one for which the most blocked holes were found (85%) and for which 22% remained blocked after the cleaning and rinsing of the nails.

This first result shows that the galenic form and in particular the viscosity has an influence on the obstruction of the holes and therefore potentially on the diffusion of terbinafine.

After 5 days of application, the results describing the amount of terbinafine that has diffused in the receiving liquid are in three groups:
the amphoteric solution has an accumulated amount of around 2.3 mg·cm⁻²;
the Lamisil Spray has an accumulated amount of around 0.3 mg·cm⁻²;
the 3 other formulations, Lamisilate Monodose, Lamisil Gel and Lamisil Cream, for which the accumulated amount is between 0.015 and 0.04 mg·cm⁻².

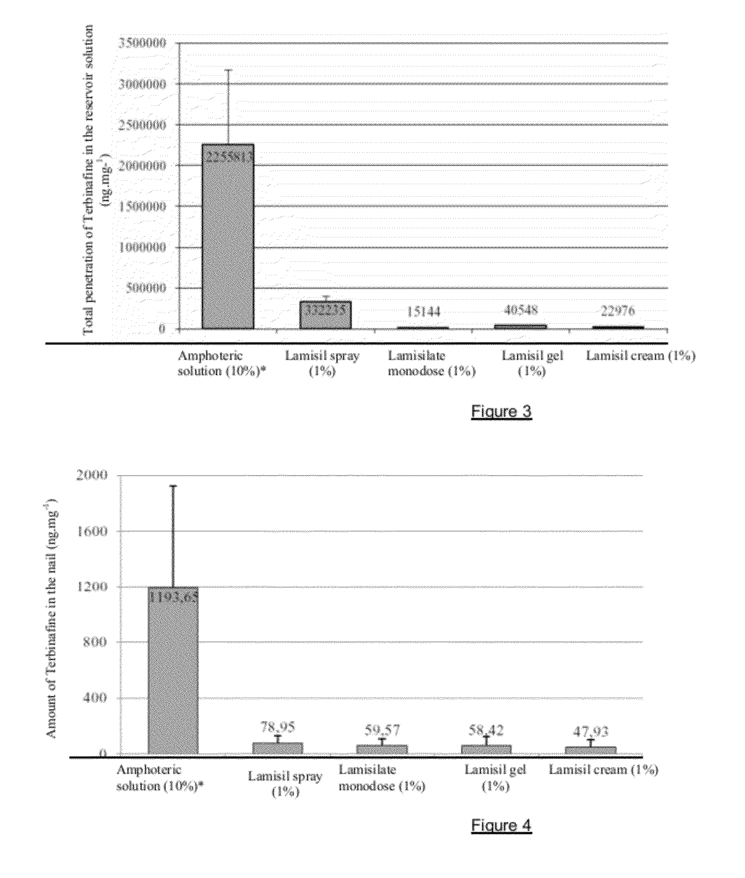

The various amounts of terbinafine accumulated in the receiving liquid are represented in FIG. 3.

The amphoteric solution has the largest amount of terbinafine accumulated in the receiving liquid in so far as it has the strongest concentration, 10%. On the other hand, at equivalent concentration (1%), the Lamisil Spray has the largest accumulated amount of terbinafine. This result can be directly correlated, on the one hand, to the viscosity and, on the other hand, to the blocking of the holes. Indeed, the Lamisil Spray has a much lower viscosity compared to those of the Lamisil Gel and the Lamisil Cream respectively. Furthermore, not having blocked the holes unlike the other formulations, the Lamisil Spray enabled the diffusion of terbinafine into the receiving liquid.

The amount of terbinafine found in the nail was measured at the end of the study. The data obtained is given in FIG. 4. The greatest concentration of terbinafine is obtained with the amphoteric solution. For this solution, the average concentration in the nail is around 1200 ng·mg⁻¹. The 4 other formulations have quite similar terbinafine concentrations, between 45 and 75 ng·mg⁻¹.

Taking the amphoteric solution as a reference, that is to say with a concentration normalized to 1%, the largest amount of terbinafine that has diffused into the receiving liquid is obtained with the amphoteric solution and the Lamisil Spray. For these two solutions, the amount of terbinafine found in the receiving liquid varies on average between 9 and 11% of the dose applied (FIG. 5). With the other Lamisilate Monodose, Lamisil Gel and Lamisil Cream formulations, only 1% of the dose of terbinafine applied is found in the receiving liquid.

### EXAMPLE 7

### Study of the Diffusion of Terbinafine Through Perforated Nails

The objective of this study is to study the diffusion of terbinafine HCl through perforated nails, by comparing two solutions with and without amphoteric agent, prepared according to the process described in Example 1:

Solution with 10% w/w terbinafine HCl amphoteric agent according to Example 3;

Solution without 10% w/w terbinafine HCl amphoteric agent that has the same composition as Example 3 except that the amphoteric agent is replaced by an equivalent amount of water.

### A/ Material and Methods:

The formulations are evaluated in duplicate on perforated cadaver nails from different donors, except for the thumb. Amounts of 10 μl/cm² are applied daily for 5 days, the nails being first fastened to diffusion cells.

The protocol of the study is then identical to that described in Example 4.

### B/ Results:

The table below represents the amount of terbinafine HCl in the receiving liquid (μg/cm²) after 5 days of application.

| | 10% w/w | 10% w/w |
|---|---|---|
| Time | amphoteric solution | solution without |
| (days) | (according to Example 3) | amphoteric agent |
| 1 | 148.9 ± 85.6 | 138.5 ± 63.6 |
| 2 | 273.7 ± 164.6 | 244.8 ± 83.5 |
| 3 | 401.1 ± 240.6 | 333.4 ± 116.7 |
| 4 | 569.5 ± 356.9 | 449.9 ± 169.4 |
| 5 | 713.2 ± 385.9 | 552.7 ± 198.1 |

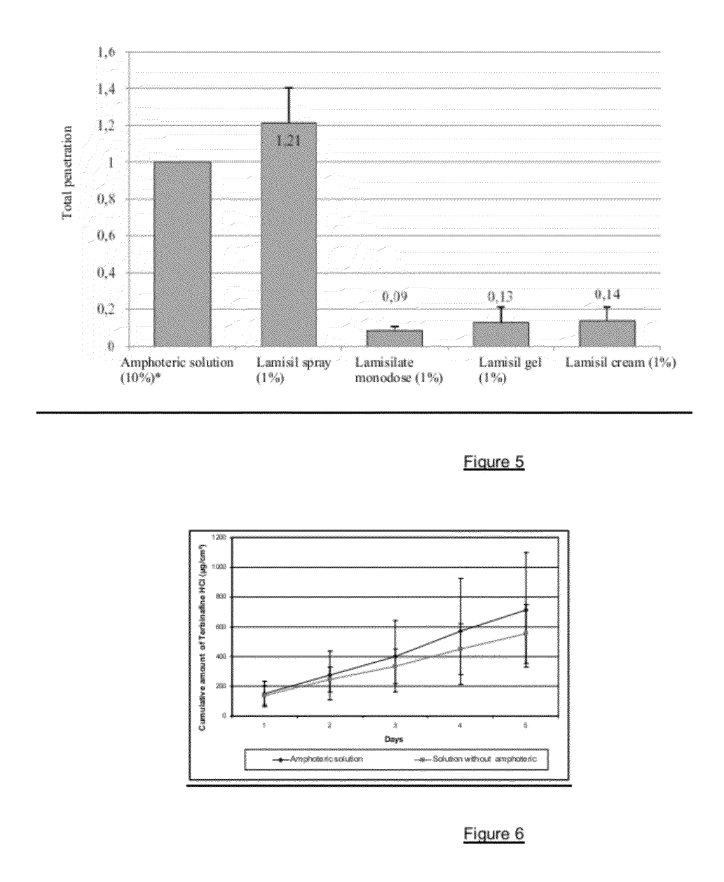

Furthermore, the amount of terbinafine accumulated in the receiving liquid (μg/cm²) through the perforated nails is illustrated in FIG. 6.

This study shows that terbinafine HCl diffuses through the perforated nail better when the composition contains amphoteric agent.

### EXAMPLE 8

### Study of the diffusion of a Dye (Nile Red) Through Perforated Nails

The objective of this study is to evaluate, on perforated nails, the diffusion of a dye, Nile Red, present in a solution with and without amphoteric agent.

The formulations tested correspond to that from Example 3 in which:

for the amphoteric solution, the terbinafine HCl was replaced by 0.03% Nile Red;

for the solution without amphoteric agent, the terbinafine HCl was replaced by 0.03% Nile Red and the amphoteric agent by an equivalent amount of water.

### A/ Material and Methods:

The experiment relates to nails of human cadavers perforated with 3 holes and placed in Franz type diffusion cells. 2 ml of the formulation to be tested is applied to each nail.

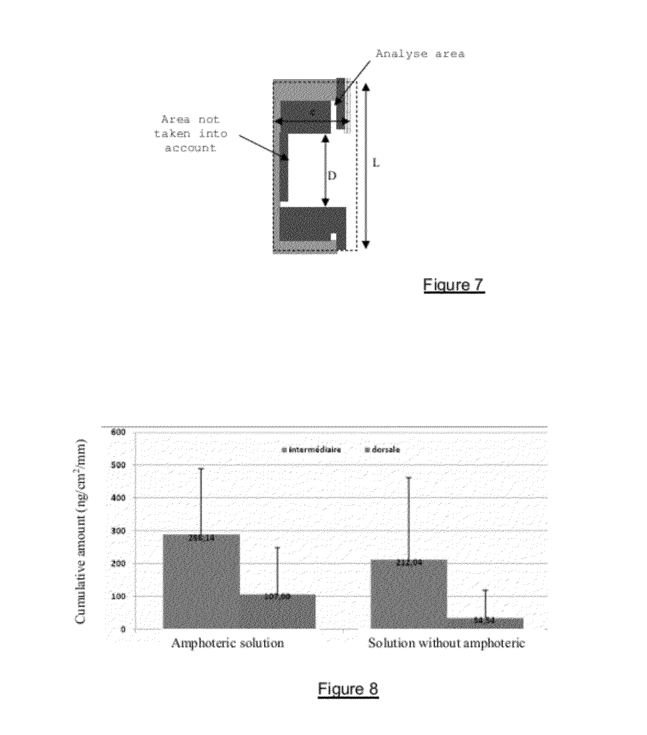

The diffusion of Nile Red is monitored for 24 hours in the absence of light and at ambient temperature. At the end of the study the nails are rinsed thoroughly with demineralised water. The nails are then cut up transversally and the analysis of the distribution of the dye in the nail takes place via observation of the slice of nail using a confocal microscope equipped with acquisition software and image processing software. The zones to be taken into account for the analysis are presented in FIG. 7.

### B/ Results:

Analysis of the coloured images shows that the diffusion zone on the side walls of the holes is larger than that obtained from the dorsal zone, that is to say the upper part of the nail. The table below presents the percentage of surface coloured as a function of the formulation tested.

| | Solution without | Amphoteric |
|---|---|---|
| | amphoteric agent | solution |
| Surface coloured (%) | 11.01 | 21.13 |

This study demonstrates that, as a function of the presence of the amphoteric agent in the composition:

the diffusion into the nail of the Nile Red dye is greater or lesser;

the diffusion of the dye on the side walls of the hole corresponding to the intermediate layer of the nail is greater in comparison with the dorsal layer.

Thus, as a function of the dorsal or intermediate layer of the nail, the diffusion of Nile Red is different. This result therefore highlights that the nature of the layer of the nail has an impact on the diffusion of the dye.

Furthermore, as a function of the presence or absence of the amphoteric agent in the solution as prepared, the diffusion of the dye is different. This diffusion is even greater when the amphoteric agent is present in the composition.

### EXAMPLE 9

### Study of the Wettability of the Various Layers of the Nail

The objective of this study is to characterize the surface properties of the various layers of the nail (dorsal, intermediate, ventral) by determining their respective surface tensions.

### A/ Material and Methods:

The experiments are carried out on nails from human cadavers. The contact angle was measured using a goniometer (Contact angle measuring system G10, KRÜSS, Germany). The surface tension of the various layers of the nail was determined by the two-liquids method using reference solutions: water and diiodomethane.

The measurements begin on the dorsal face of the nail. The measurements are carried out at room temperature. A drop (˜2-5 μl) of demineralised water is deposited on the dorsal face of the nail using a needle and a syringe positioned vertically at the surface of the nail. The drop thus in contact on the nail is photographed. The software then measures the contact angle that the drop makes with the surface of the nail. The drop is then wiped with filter paper. The same procedure (depositing the drop, measuring the contact angle) is repeated 10 times in a row for the solution tested. The surface of the nail is rapidly cleaned with ethanol. Identical measurements are then carried out successively with diiodomethane.

Once the measurements on the dorsal layer are finished, the nail is recovered and ground down in order to carry out the measurements on the intermediate layer as described previously. Once all the measurements are finished, the nail is turned over in order to carry out contact angle measurements on the ventral face with the 2 solutions.

### B/ Results:

The averages of the surface tensions (γ_{S}) of the 3 layers of the nail (mJ.m⁻²) are given below:

| Layer of the nail | γₛ (mJ · m⁻²) |
|---|---|
| dorsal | 33.52 ± 6.16 |
| intermediate | 44.36 ± 3.40 |
| ventral | 54.71 ± 6.28 |

These results introduce new elements with respect to the wettability of the various layers of the nail. Specifically, these results show that the nail is not a homogeneous biological material with respect to this parameter. Each layer has its own wettability properties that are quite different from the other layers.

The dorsal layer represents a barrier in terms of wettability with respect to the other two layers which are more wettable due to their surface tension being higher than that of the dorsal layer.

These results are in accordance with the preceding results that show a difference in diffusion of the Nile Red dye as a function of the nature of the layer.

### EXAMPLE 10

### Study of the Diffusion of Terbinafine HCl Through the Various Layers of the Nail:

The objectives of this study are:

to study the influence of the nature of the layer of the nail on the diffusion of terbinafine HCl due to the difference in wettability of the dorsal and intermediate layers,

to study the influence of the amphoteric agent on the diffusion of terbinafine HCl through the various layers of the nail.

The formulation containing amphoteric agent corresponds to that from Example 2.

The formulation without amphoteric agent corresponds to that from Example 2 in which the amphoteric agent has been replaced with an equivalent amount of water.

For each formulation, 2 nails (1 with the dorsal layer and 1 without the dorsal layer) are used per donor. The treatment time is 5 days with a new application of each formula every day. The amount of formula deposited daily on the surface of the nail is 10 μl/cm2.

### A/ Material and Methods:

For each donor, the thickness of the nails is measured. Then, for each donor, the nails are prepared in the following manner:

4 nails without dorsal layer: for the nails from this group, the dorsal face is removed using a miniature sander in order to reach the intermediate layer by removing 40% of the initial thickness of the nail,

4 nails with dorsal layer: for the nails from this group, the ventral face is taken off using a miniature sander in order to reach the intermediate layer by removing 40% of the initial thickness of the nail.

The Franz type diffusion cells used are placed in a thermostatically-controlled bath in order to obtain a temperature of 32±1° C. at the surface of the nail. The nails are placed in the diffusion cell with the ventral side towards the receiving compartment, phosphate buffer (pH 7.4±0.1) containing 0.1% of Volpo (Oleth-20). During the experiment, the diffusion cells are placed in a thermostatically-controlled bath in order to obtain a temperature of 32±1° C. This temperature is checked before each withdrawal. For all the nails, the application of the formula (10 μl/cm²) is repeated every 24 h for 5 days.

The protocol for the study is then identical to that described in Example 4.

The samples, receiving liquids and nail extracts are then analysed by MS/MS.

### B/ Results:

To get round the variability in the thickness of the nail, the accumulated amounts of terbinafine HCl in the receiving liquid (ng/cm²) were normalized. The results are therefore expressed in ng/cm²/mm in the table below.

| Time | 10% Solution with amphoteric agent Intermediate layer | | | | | | Average |
|---|---|---|---|---|---|---|---|
| (d) | Cell 1 | Cell 2 | Cell 3 | Cell 4 | Cell 5 | Cell 6 | (ng/cm²/mm) |
| 0 | 0.00 | 0.00 | 0.00 | 0.00 | 0.00 | 0.00 | 0.00 |
| 5 | 57.98 | 154.35 | 238.15 | 392.94 | 253.76 | 631.67 | 288.14 ± 210.77 |
| | 10% Solution with amphoteric agent Dorsal layer | | | | | | Average |
| | Cell 1 | Cell 2 | Cell 3 | Cell 4 | Cell 5 | Cell 6 | (ng/cm²/mm) |
| 0 | 0.00 | 0.00 | 0.00 | 0.00 | 0.00 | 0.00 | 0.00 |
| 5 | 14.63 | 389.43 | 55.44 | 20.50 | 79.55 | 82.44 | 107.00 ± 141.28 |
| | 10% Solution without amphoteric agent Intermediate layer | | | | | | Average |
| | Cell 1 | Cell 2 | Cell 3 | Cell 4 | Cell 5 | Cell 6 | (ng/cm²/mm) |
| 0 | 0.00 | 0.00 | 0.00 | 0.00 | 0.00 | 0.00 | 0.00 |
| 5 | 0.00 | 126.73 | 686.15 | 239.34 | 21.50 | 198.53 | 212.04 ± 250.70 |
| | 10% Solution without amphoteric agent Dorsal layer | | | | | | Average |
| | Cell 1 | Cell 2 | Cell 3 | Cell 4 | Cell 5 | Cell 6 | (ng/cm²/mm) |
| 0 | 0.00 | 0.00 | 0.00 | 0.00 | 0.00 | 0.00 | 0.00 |
| 5 | 0.00 | 0.00 | 0.00 | 0.00 | 0.00 | 207.23 | 34.54 ± 84.60 |

Thus, the amount of terbinafine accumulated relative to the thickness of the nails (ng/cm²/mm) after 5 days of application is illustrated in FIG. 8.

It appears that:

With or without amphoteric agent, the diffusion of terbinafine is greater through the intermediate layer compared to the dorsal layer;

The presence of the amphoteric agent promotes the penetration of terbinafine (×3) through the dorsal layer. On the other hand, the action of the amphoteric agent on the intermediate layer is less significant although there is a tendency to increase the diffusion through the intermediate layer too.

The amounts of terbinafine HCl diffused through the dorsal and intermediate layers are greater when the formulation contains the amphoteric agent.

These results clearly show that the dorsal layer is a barrier to the penetration of terbinafine HCl through the nail. Once this barrier is cleared, the amounts of terbinafine HCl that have diffused after 5 days of application are greater.

The amounts of terbinafine HCl in the nail (ng/mg) are given in the table below:

| Time | 10% Solution with amphoteric agent Intermediate layer | | | | | | Average |
|---|---|---|---|---|---|---|---|
| (d) | Cell 1 | Cell 2 | Cell 3 | Cell 4 | Cell 5 | Cell 6 | (ng/mg) |
| 5 | 1428.41 | 1583.09 | 1982.11 | 1354.79 | 1517.44 | 1176.74 | 1507.10 ± 272.04 |
| | 10% Solution with amphoteric agent Dorsal layer | | | | | | Average |
| | Cell 1 | Cell 2 | Cell 3 | Cell 4 | Cell 5 | Cell 6 | (ng/mg) |
| 5 | 2806.07 | 3180.52 | 2770.33 | 2122.09 | 2177.24 | 1544.53 | 2433.46 ± 593.84 |
| | 10% Solution without amphoteric agent Intermediate layer | | | | | | Average |
| | Cell 1 | Cell 2 | Cell 3 | Cell 4 | Cell 5 | Cell 6 | (ng/mg) |
| 5 | 718.20 | 1796.26 | 1506.90 | 1263.62 | 2238.02 | 1899.68 | 1570.45 ± 534.70 |
| | 10% Solution without amphoteric agent Dorsal layer | | | | | | Average |
| | Cell 1 | Cell 2 | Cell 3 | Cell 4 | Cell 5 | Cell 6 | (ng/mg) |
| 5 | 2852.58 | 2163.81 | 2569.12 | 5402.01 | 2431.90 | 2784.16 | 3033.93 ± 1186.48 |

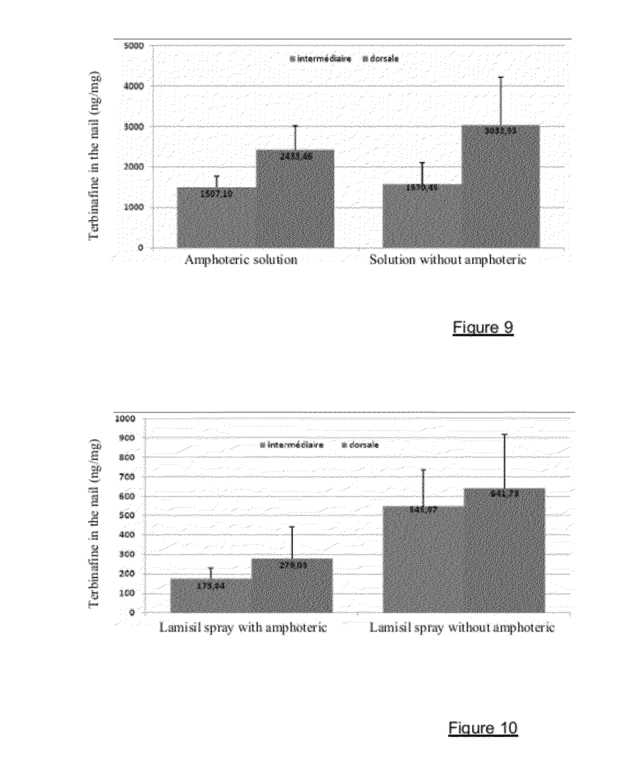

Furthermore, the amount of terbinafine HCl in the nail (ng/mg) is illustrated in FIG. 9.

It appears that:

With or without amphoteric agent, the amount of terbinafine HCl found in the nail is greater in the dorsal layer than in the intermediate layer.

The presence of the amphoteric agent makes it possible to reduce the reservoir effect of the molecule in the dorsal layer by reducing the amount of terbinafine HCl stored in this layer.

On the other hand, the effect of the amphoteric agent is less pronounced in the intermediate layer due to a short application time, only 5 days, and a high concentration 10% (w/w) of terbinafine HCl.

In order to confirm the result according to which the amphoteric agent makes it possible to promote the diffusion of the terbinafine HCl through the nail, both through the dorsal layer and through the intermediate layer, this same diffusion study was carried out with the commercial formula Lamisil Spray 1% with and without amphoteric agent.

The procedures of the study and also the amounts of terbinafine HCl in the various layers of the nail, dorsal and intermediate, were carried out and analysed according to the same protocol as before.

The amounts of terbinafine HCl in the nail (ng/mg) are given in the table below:

| Time | 1% Lamisil Spray with amphoteric agent Intermediate layer | | | | | | Average |
|---|---|---|---|---|---|---|---|
| (d) | Cell 1 | Cell 2 | Cell 3 | Cell 4 | Cell 5 | Cell 6 | (ng/mg) |
| 5 | 149.83 | 153.47 | 247.22 | 231.39 | 165.44 | 95.69 | 173.84 ± 56.33 |
| | 1% Lamisil Spray with amphoteric agent Dorsal layer | | | | | | Average |
| | Cell 1 | Cell 2 | Cell 3 | Cell 4 | Cell 5 | Cell 6 | (ng/mg) |
| 5 | 184.04 | 256.60 | 157.75 | 196.22 | 283.56 | 596.00 | 279.03 ± 162.22 |
| | 1% Lamisil Spray Intermediate layer | | | | | | Average |
| | Cell 1 | Cell 2 | Cell 3 | Cell 4 | Cell 5 | Cell 6 | (ng/mg) |
| 5 | 615.72 | 475.81 | 541.52 | 884.45 | 351.10 | 407.24 | 545.97 ± 190.58 |
| | 1% Lamisil Spray Dorsal layer | | | | | | Average |
| | Cell 1 | Cell 2 | Cell 3 | Cell 4 | Cell 5 | Cell 6 | (ng/mg) |
| 5 | 502.52 | 498.12 | 1022.72 | 969.48 | 438.16 | 419.40 | 641.73 ± 276.93 |

The amount of terbinafine HCl in the nail (ng/mg) is illustrated in FIG. 10.

It appears that:

As for the amphoteric solution, the amount of terbinafine HCl is greater in the dorsal layer than in the intermediate layer.

The presence of the amphoteric agent makes it possible to very significantly reduce (by a factor of 3) the amount of terbinafine HCl both in the dorsal layer and in the intermediate layer.

Due to a smaller amount of terbinafine HCl in the composition, the effect of the amphoteric agent on the reduction in the storage of the active principle in the two layers is less pronounced.

### CONCLUSIONS

In conclusion, the various results therefore show that:

The compositions must have particular specifications in terms of content of volatile solvent (except for water) and of viscosity in order to enable the formulation, on the one hand, to penetrate into the holes without obstructing them and, on the other hand, to enable the terbinafine HCl to diffuse into the intermediate layer.

The nail, as a function of the nature of the layers, dorsal, intermediate and ventral, has different surface properties that are expressed by a different surface tension. Specifically, the dorsal layer is substantially less wettable than the intermediate and ventral layers.

The comparative diffusion studies of terbinafine HCl through the dorsal layer and the intermediate layer have shown that the intermediate layer facilitates the diffusion of the active principle. This result may be correlated to a greater wettability of the intermediate layer relative to the dorsal layer.

The presence of the amphoteric agent facilitates the diffusion of terbinafine HCl through the dorsal layer and the intermediate layer.

The presence of the amphoteric agent reduces the storage of terbinafine HCl in the dorsal layer and the intermediate layer, which facilitates its diffusion.

Thus, the creation of holes through the nail plate gives access to the intermediate layer, which is more favourable to the diffusion of terbinafine HCl than the dorsal layer.

Due to the presence of the amphoteric agent in the composition, the terbinafine HCl diffuses much better in the intermediate layer, by a factor of 3 relative to the dorsal layer. The terbinafine HCl diffuses better through the pierced nail when the composition contains amphoteric agent.

The presence of holes therefore makes it possible to:
on the one hand, reach the nail bed directly without passing through the nail plate,
on the other hand, reach the nail bed via the intermediate layer with larger amounts.

The combined effect of these two aspects makes it possible to obtain higher effective concentrations of terbinafine HCl at the nail bed.

1. A pharmaceutical composition intended to be applied to a perforated nail, the composition comprising:
an antifungal agent that is in the form of an acid salt; and
a solvent system;
said composition comprising a cationic surfactant and said composition having a viscosity of less than 500 cPs, said viscosity being measured according to the Brookfield method at 25° C.

2. The composition as defined by claim 1, wherein the solvent system is comprised of volatile solvent(s), except for water, of less than or equal to 40% by weight of the total composition.

3. The composition as defined by claim 1, wherein the amphoteric surfactant is a derivative of betaine, corresponding to the general formula (I) below: 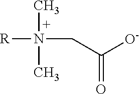
in which R represents an alkyl radical or an R′CO—NH(CH₂)₃— radical, R′ representing an alkyl radical.

4. The composition as defined by claim 3, wherein the amphoteric surfactant is coco betaine.

5. The composition as defined by claim 3, wherein the surfactant represents from 0.1% to 20% by weight of the composition.

6. The composition as defined by claim 1, wherein the antifungal agent is an allylamine or morpholine.

7. The composition as defined by claim 1, wherein the antifungal agent represents at least 5%, by weight of the total composition.

8. The composition as defined by claim 1, wherein the solvent system is an aqueous system comprised of:
water;
at least one C2-C8 alkanol with a straight or branched chain, advantagcously cthanol; and
at least one glycol,

9. The composition as defined by claim 1, wherein the composition also comprises a texturing agent.

10. The composition as defined by claim 1, wherein the composition comprises at least one compound selected from the group consisting of a chelating agent, an antioxidant, an antiseptic, and an emollient.

11. A method of preparing a medicament, the method comprising preparing the medicament with an effective amount of the composition according to claim 1, and formulating the medicament for application to a perforated nail for treating onychomycoses.

12. The method as defined by claim 11, wherein the nail has holes with a diameter from 400 μm to 1 mm.

13. The method as defined by claim 11, wherein the nail has holes with a depth that represents from 10% to 100% of the thickness of the nail.

14. The composition as defined by claim 1, wherein the antifungal agent is a hydrochloride.

15. The composition as defined by claim 1, wherein the cationic surfactant is a positively charged amphoteric surfactant.

16. The composition as defined by claim 1, wherein the viscosity of the composition is from 300 cPs to 150 Cps.

17. The composition as defined by claim 1, wherein the surfactant represents from 0.1% to 15% by weight of the composition.

18. The composition as defined by claim 6, wherein the surfactant represents from 0.1% to 10% by weight of the composition.

19. The composition as defined by claim 6, wherein the antifungal agent is terbinafine or the hydrochloride thereof.

20. The composition as defined by claim 7, wherein the antifungal agent represents at least 8% by weight of the total composition.

21. The composition as defined by claim 7, wherein the antifungal agent represents at least 10% weight of the total composition.

22. The composition as defined by claim 8, wherein the at least one C2-C8 alkanol is ethanol.

23. The composition as defined by claim 8, wherein the at least one glycol is propylene glycol.

24. The composition as defined by claim 9, wherein the texturing agent is an alkyl cellulose.

25. The composition as defined by claim 9, wherein the texturing agent is present at a content of 0.3% to 0.5%.

26. The method as defined by claim 12, wherein the diameter of the holes in the nail is from 400 μm to 600 μm.

27. The method as defined by claim 12, wherein the holes in the nail have a depth of from 0.2 mm to 5 mm.
